(19) United States
(12) Patent Application Publication
(10) Pub. No.: US 2024/0274036 A1
(43) Pub. Date: Aug. 15, 2024

(54) VENOUS ACCESS SIMULATION DEVICE

(71) Applicant: ORLANDO HEALTH, INC., Orlando, FL (US)

(72) Inventors: Shveta D. Gupta, Orlando, FL (US); Keely DeGroot, Orlando, FL (US); Stephanie Sharon, Orlando, FL (US); Jack Stubbs, Orlando, FL (US)

(73) Assignee: ORLANDO HEALTH, INC., Orlando, FL (US)

(21) Appl. No.: 18/643,791

(22) Filed: Apr. 23, 2024

Related U.S. Application Data
(63) Continuation of application No. PCT/US2023/071201, filed on Jul. 28, 2023.
(60) Provisional application No. 63/393,629, filed on Jul. 29, 2022.

(51) Int. Cl. G09B 23/28; B33Y 10/00; B33Y 80/00; G09B 23/30

(52) U.S. Cl. CPC G09B 23/285; B33Y 10/00; B33Y 80/00; G09B 23/303

(57) ABSTRACT

A medical simulation device for providing interactive training for blood vessel identification and venous access is disclosed. The medical simulation device includes a tissue simulation surface, a reservoir configured to contain a fluid, and a channel in fluid communication with the reservoir. The channel extends from the reservoir and passes beneath the tissue simulation surface and is accessible to receive a needle inserted through the tissue simulation surface. The tissue simulation surface is formed to simulate an anatomical tissue surface and provide realistic tactile responsiveness and feedback such that the channel simulates a blood vessel within the anatomical tissue. The medical simulation device can provide a realistic training experience for users to learn the proper techniques of administering intravenous injections, blood draws, and insertion of catheters.

### CROSS-REFERENCE TO RELATED APPLICATIONS

This application is a Continuation of PCT/US2023/071201, titled VENOUS ACCESS SIMULATION DEVICE, filed on Jul. 28, 2023, and published as WO 2024/026451 A2, that claims the benefit of U.S. Provisional Application No. 63/393,629, titled INTRAVENOUS ACCESS SIMULATION DEVICE, filed on Jul. 29, 2022; the entirety of each are incorporated herein by reference.

### TECHNICAL FIELD

The present disclosure relates to a medical simulation device and method for manufacturing the same, and more specifically, relates to a venous access simulation device used to provide interactive training for various medical operations, including blood vessel palpations, blood draws, intravenous injections, and catheter insertions.

### BACKGROUND

Venous catheters are a commonly used medical device inserted into an individual's veins to perform various medical administrations, tests, or procedures, including for taking blood samples, providing fluids or blood, performing medical tests, and administering medication. There are many different types of venous catheters, including central venous catheters, also known as a central line or CVC, peripherally inserted central venous catheter, also known as a PICC line, and peripheral intravenous catheter, also known as a peripheral IV or standard IV.

Venipuncture, including the insertion of needles to perform blood draws and intravenous injections, is also a very common medical operation performed throughout the world. Intravenous injection is the injection of a medication of a medication or other substance into a vein and directly into the bloodstream using a needle and/or catheter. Blood draws are the insertion of a needle into a blood vessel to take blood from a vein, usually for laboratory testing. Learning to properly administer intravenous injections and blood draws often requires substantial practice, as veins are not easily visible through the skin. To properly locate a vein to insert an intravenous needle, often an individual must palpate the skin. Learning proper palpation techniques often requires substantial practice and training.

Medical professionals receive training of administering catheters, including central venous catheter, peripherally inserted central venous catheter, peripheral intravenous venous catheters, intravenous injections, and blood draws through formal education or on-the-job training. Individuals administering at-home therapy may also require training of administering intravenous injections and blood draws. Such training may include the use of medical simulation devices. Prior approaches to train in performing venous access procedures on simulation devices include use of benchtop simulators, mannequins, or simulated body parts. These devices are often are not very adaptable to simulate a variety of patient variables, are not readily producible, wear out quickly, are large or require complicated setups that make them expensive and undesirable for training medical professionals or training at-home patients. Additionally, many of these prior approaches include unrealistic or bulky simulated body parts that do not permit a user to practice and experience administering catheter insertions or intravenous injections with the device on their own body or simulated body part with ease and is also readily producible and configurable. The embodiments described in this application address these shortcomings.

### BRIEF DESCRIPTION OF THE DRAWINGS

The embodiments discussed herein may be better understood with reference to the following drawings and description. The components in the figures are not necessarily to scale. Moreover, in the figures, like-referenced numerals designate corresponding parts through the different views and embodiments.

FIG. 1 is a perspective view of a first embodiment of a 3D printed medical simulation device in accordance with certain aspects of the present disclosure.

FIG. 2 is a perspective view of a second embodiment of a medical simulation device in accordance with certain aspects of the present disclosure.

FIGS. 3A and 3B are depictions of a third embodiment of a medical simulation device in accordance with certain aspects of the present disclosure.

FIG. 4 is an illustration of a fourth embodiment of a medical simulation device in accordance with certain aspects of the present disclosure.

FIGS. 5A and 5B are depictions of a fifth embodiment of a medical simulation device in accordance with certain aspects of the present disclosure.

FIG. 6 is a flow diagram of an example method for the manufacture of a three-dimensional printed device.

FIGS. 7A-7C are depictions of a non-limiting prototype of a medical simulation device in accordance with certain aspects of the present disclosure.

FIGS. 8A-8C are depictions of a sixth embodiment of a medical simulation device in accordance with certain aspects of the present disclosure.

FIG. 9 is a depiction of an example setup of the medical simulation device as described with reference to FIGS. 8A-8C.

FIG. 10A-10C are detailed depictions of a component of the medical simulation device in accordance with certain aspects of the present disclosure.

### DETAILED DESCRIPTION OF THE DRAWINGS

This disclosure describes example implementations of a venous access simulation device used to provide interactive training for various medical operations, including blood vessel palpations, blood draws, intravenous injections, peripheral venous catheter insertions, peripherally inserted central venous catheters, and central venous catheter insertions.

Various aspects are described below with reference to the drawings in which like elements generally are identified by like numerals. The relationship and functioning of the various elements of the aspects may be better understood by reference to the following detailed description. However, aspects are not limited to those illustrated in the drawings or explicitly described below. It should be understood that other embodiments may be utilized and structural changes may be made without departing from the scope of the invention. It should also be understood that the drawings are not necessarily to scale, and in certain instances, details may have been omitted that are not necessary for an understanding of aspects disclosed herein, such as conventional manufacturing and assembly techniques.

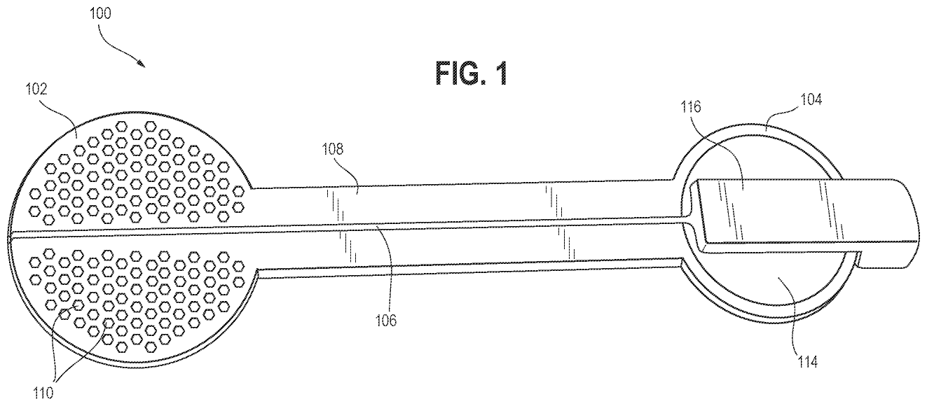

FIG. 1 is a perspective view of a first embodiment of a medical simulation device 100 in accordance with certain aspects of the present disclosure. Medical simulation device 100 may be produced using additive manufacturing, and in particular, 3D printing. All, or portions of, medical simulation device 100 may also be produced using conventional manufacturing processes, including forming, molding, or other suitable process. Medical simulation device 100 includes a tissue simulation structure 102, a compressible reservoir 104, and a channel 106 in fluid communication with the compressible reservoir 104. Medical simulation device 100 may be used to provide training for various medical operations, including intravenous injections, blood draws, and insertion of catheters, including peripherally inserted central line catheters, by simulating a blood vessel underneath the skin. Although not depicted in FIG. 1, medical simulation device 100 may include an additional layer of material covering what is shown. The additional layer of material on the top of the tissue simulation structure 102 may form a tissue simulation surface for simulating an anatomical tissue surface. Medical simulation device 100 allows a user to learn the proper techniques of blood vessel palpation and intravenous injection administration by applying an external pressure to the compressible reservoir 104, palpate the tissue simulation surface to locate the simulated blood vessel, i.e., channel 106, and insert a needle into the simulated blood vessel.

Medical simulation device 100 includes a tissue simulation structure 102. The tissue simulation structure 102 may be formed via 3D printing. For example, and without limitation, processes from one or more of the following patents may be utilized, each following patent application hereby incorporated by reference in its entirety: U.S. Pat. Nos. 10,860,001, 10,649,440, 10,558,199, 10,379,525, and 10,073,440. In certain embodiments, the tissue simulation structure 102 may be 3D printed to have a texture that is similar to the human body. For example, the tissue simulation structure 102 may be formed such that it feels and looks like human skin tissue, muscle tissue, fat tissue, or subcutaneous tissue. In order to achieve such a texture, the tissue simulation structure 102 may be composed of printed 3D material and fluid-filled pockets 110. The fluid-filled pockets 110 may be filled with air or other suitable gas, or may be filled with liquid, e.g., water, saline solution, etc. The combination of 3D printed material and fluid-filled pockets 110 forms a composite structure of tissue simulation structure 102.

The composite structure forming tissue simulation structure 102 is designed to simulate the feel and tactile response of human skin tissue. The properties of the composite structure may be varied to simulate various types or areas of human tissue. For example, the properties of tissue simulation structure 102 may be varied to simulate the tissue on the back of the hand, on the forearm, on the inside of the elbow, or other locations where intravenous injections are administered. The properties of the composite structure may also be varied such that the simulated tissue is similar to the tissues of a specific person. For example, the material properties of tissue simulation structure 102 may be softer, i.e., less stiff, to be more similar to, and simulate, a person with softer skin tissues, or may be harder, i.e., relatively high degree of stiffness, to be more similar to, and simulate, a person with harder skin tissues. The material properties of tissue simulation structure 102 may also be varied to simulate other tissue and blood vessel properties. For example, the material properties of tissue simulation structure 102 may be varied to simulate different levels of vein roll. Thus, to simulate a person who is more prone to vein roll, the tissue simulation structure 102 may be softer, i.e., less stiff.

The tissue simulation structure 102 may be formed using one or more base materials and bodies to generally match the tactile responsiveness and other properties of a desired skin tissue. The base materials can be independent or can be blended via material blending to provide a realistic tissue structure. Various parameters may be tuned to best replicate the tactile responsiveness and other properties of the desired body tissue. The tissue simulation structure 102 may be formed using one or more various materials including, for example, an elastomeric material such as an organic or silicone-based molecule, latex, polymeric material, plastic, gel, and/or viscous fluid, a two-part urethane, a semi-rigid material such as polyethylene, or other suitable material. The material may be selected to have a density and/or other properties that are similar to a corresponding anatomical tissue. In some examples, the tissue simulation structure 102 may include a fibrous layer, such as a nylon mesh to correspond to anatomical tissue. In some examples, some portion of the tissue simulation structure 102 may include a hollow or open space portion.

While not depicted in FIG. 1, tissue simulation structure 102 may include a top layer of material forming a simulation surface. The simulation surface may be formed such that it covers channel 106. The simulation structure may also cover the fluid-filled pockets 110, such that the fluid within the fluid-filled pockets 110 is sealed and contained within the medical simulation device 100. The tissue simulation surface may be formed of the same materials as tissue simulation structure 102, or may be of a different material. For example, the tissue simulation surface may be formed such that it matches the properties of the outer layer of skin tissue, i.e., the epidermis, while the tissue simulation structure 102 is formed such that it matches the properties of deeper layers of skin tissue, i.e., the dermis.

The tissue simulation surface is formed and made of materials such that it simulates an anatomical tissue surface. The tissue simulation surface may also be referred to as an interactive operative area. The tissue simulation surface is formed to have a realistic skin tissue appearance and function. As discussed previously, the tactile responsiveness of the tissue simulation structure 102 may be altered. Similarly, the properties of the tissue simulation surface may also vary to simulate different skin surface types. For example, to simulate a person of thicker skin, the tissue simulation surface may be formed such that it is harder, i.e., relatively high degree of stiffness. The tissue simulation surface accordingly may be formed to provide a realistic force for insertion of a needle, and provide tactile feedback to a user. By doing so, the medical simulation device 100 provides a simulator with a skin tissue responsiveness that will generally match that of the actual patient-user's body. The user may practice using the medical simulation device 100, and experience a realistic tactile response similar to their actual body's response to practice self-administering intravenous injections.

The tissue simulation surface and tissue simulation structure 102 may be formed such that they can self-seal. Thus, if the tissue simulation surface is pierced by a needle, after the needle is withdrawn, the tissue simulation surface and tissue simulation structure 102 may seal such that no fluid escapes. Thus, even if a user unintentionally inserts a needle through the tissue simulation surface, misses the simulated blood vessel (channel 106), and pierces a fluid-filled pocket 110, the tissue simulation surface and tissue simulation structure 102 may seal to prevent any fluid loss from the fluid-filled pocket 110.

Self-sealing of the simulation structure and tissue simulation structure 102 enables repeated practice using medical simulation device 100. By self-sealing, the medical simulation device 100 may be used multiple times without degradation or visual defects. For example, by self-sealing, a user may not be able to view where the needle was previously inserted. This may help ensure that a user cannot simply use visual indicators of previous insertion sites to determine the location of the simulated blood vessel. Instead, the user learns to employ proper palpation techniques for locating and inserting intravenous needles into blood vessels.

The properties of the tissue simulation surface and/or tissue simulation structure 102 may also be varied to match different color tones. For example, the color of the tissue simulation surface and/or the tissue simulation structure 102 may be matched to the skin tone of a particular patient and/or user. Matching the color of tissue simulation surface and/or the tissue simulation structure 102 to a patient-user's skin tone may help increase the training effectiveness of the medical simulation device 100. By matching the color of the tissue simulation surface and/or tissue simulation structure 102 to the patient-user's skin, the patient-user may begin to mentally blur differences between the medical simulation device 100 and their own tissues, thus increasing the believability and effectiveness of the training. The tissue simulation surface may also be formed of a generally transparent or translucent material such that a user can visually perceive the channel 106 based on a colored fluid. This may help users begin to learn the tactile sensations of palpation and proper insertion of the needle while still being able to utilize visual indicators.

Medical simulation device 100 includes a compressible reservoir 104 that is in fluid communication with a channel 106. The compressible reservoir 104 is formed such that it can contain a fluid. The fluid may be a blood simulation fluid, water (e.g., water dyed red), saline solution, or other suitable liquid. Compressible reservoir 104 is formed of a suitable material such that it can deform and pressurize when the compressible reservoir 104 can pressurize the fluid upon application of an external pressure (i.e., pressurize the fluid above the surrounding atmospheric pressure). A tourniquet may be used to apply an external pressure to the compressible reservoir 104. For example, when the medical simulation device 100 is placed on a patient's arm, a human simulated-arm, or other suitable structure, a tourniquet may be then be wrapped around or applied to the arm or other structure and tightened and/or pressurized such that the tourniquet applies pressure to the compressible reservoir 104. In alternative embodiments, a pump (not shown) may provide such pressure, and it is contemplated that the pump may circulate fluid through the device at a particular flow rate to simulate blood flow within the patient's anatomy.

Compressible reservoir 104 may be formed via additive manufacturing, as shown in FIG. 1, or may formed via conventional manufacturing processes such as forming or molding. When formed via additive manufacturing, e.g., 3D printing, a reservoir support material 114 may be used to support the forming of the compressible reservoir 104. The reservoir support material 114 may accordingly define the internal surface of the compressible reservoir 114. Upon completion of production of the compressible reservoir 104 via additive manufacturing, the reservoir support material 114 may be removed, leaving a cavity within the compressible reservoir 104.

The compressible reservoir 104 is designed such that the volume of contained fluid is large compared to the overall volume of channel 106. Upon application of the external pressure, the compressible reservoir 104 may pressurize the fluid. Because the channel 106 is in fluid communication with the compressible reservoir 104, when the fluid in the compressible reservoir 104 is pressurized, the fluid in the channel 106 also becomes pressurized.

The compressible reservoir 104 may also include an access port that can be plugged. The access port can be used for several different purposes. For example, when the compressible reservoir is formed using additive manufacturing, the reservoir support material 114 may be removed through the access port. After filling the compressible reservoir 104 with fluid, the access port may then be permanently plugged to contain the fluid. The access port may also only be temporarily plugged. By temporarily plugging the access port, access port may allow for repeated emptying and refilling of the compressible reservoir 104 with fluid.

While in FIG. 1, the compressible reservoir 104 is shown as being affixed and rigidly attached to the tissue simulation structure 102 via channel structure 108, this is not required. Compressible reservoir 104 may be separate from, and therefore removable from, the medical simulation device 100. Accordingly, the compressible reservoir 104 may be removable from fluid communication with channel 106. Upon removing compressible reservoir 104 from the medical simulation device 100, the compressible reservoir 104 may be filled, emptied, and/or refilled.

In some examples, compressible reservoir 104 permits fluid to flow in both directions, i.e., both from compressible reservoir 104 to channel 106 and from channel 106 to compressible reservoir 104. Accordingly, upon application of a tourniquet or other external pressure to compressible reservoir 104, as the compressible reservoir 104 is pressurized, a portion of the fluid may flow out of the reservoir. Upon release of the tourniquet or other external pressure, the fluid may flow back from channel 106 towards, i.e., return, to the compressible reservoir 104 for containment. In other examples, the compressible reservoir 104 may include a one-way valve. The one-way valve may be a check valve or other suitable type of valve. The one-way valve may be oriented such that it prevents fluid from flowing back into the compressible reservoir 104. Thus, the valve may permit fluid to only flow out of the compressible reservoir 104 and into the channel 106.

As previously described, the medical simulation device 100 is used to train in the administering of intravenous injections, blood draws, and insertion of catheters. Medical simulation device 100 includes channel 106 to simulate a blood vessel, more specifically a vein. Channel 106 may be defined by the formation of channel structure 108 that is formed via additive manufacturing, e.g., 3D printing, or may be produced by conventional manufacturing processes, e.g., forming and/or molding.

As shown in FIG. 1, the channel 106 may be formed and defined by 3D printing channel support material 116 within channel structure 108. The channel support material 116 may include a tabbed portion, and an elongate portion. The tabbed portion may be within the cavity of the compressible reservoir 104, as depicted in FIG. 1, or may be standalone. Upon completion of forming the channel structure 108, the channel support material 116 may be removed (e.g., by pulling on the tabbed portion, removing the elongate portion), leaving the cavity defining the channel 106.

As shown in FIG. 1, the channel 106 may be defined by a channel structure 108. In some examples, channel structure 108 is rigidly attached to the tissue simulation structure 102, the compressible reservoir 104, or both. In some examples, channel structure 108 is flexibly attached to the tissue simulation structure 102, the compressible reservoir 104, or both.

The external shape of channel structure 108 may be a variety of different forms. For example, as shown in FIG. 1, the external shape of channel structure 108 may generally be rectangular. The external shape of channel structure 108 may also be generally tubular, e.g., concentric with the channel 106. The channel structure 108 may be formed of the same material as the tissue simulation structure 102 and/or compressible reservoir 104, but it may also be formed of a different suitable material. Optionally, the channel structure 108 may be formed of multiple materials. For example, an initial layer defining and surrounding channel 106 may be formed of a first material while the rest of the channel structure 108 may be formed of a second material. Multiple materials may also be blended to form the channel structure 108.

Channel 106 extends from compressible reservoir 104 through at least a portion of the tissue simulation structure 102 and beneath the tissue simulation surface. As described previously, the channel 106 within the tissue simulation structure 102 simulates a blood vessel, more specifically, a vein. To simulate a vein, similar to the channel structure 108, the tissue simulation structure 102 may be formed using one or more materials. For example, an initial layer defining and surrounding channel 106 may be formed of a first material while the rest of the tissue simulation structure may be formed of a second material. In such examples, the first material may be formed to generally simulate the responsiveness and feel of a blood vessel wall, while the second material may be formed to generally simulate the responsiveness of skin tissue.

As described previously, channel 106 simulates a blood vessel, allowing users to practice palpation of the simulated blood vessel within the medical simulation device 100. This allows the user to practice locating the vein. Channel 106 may also be designed to simulate vein roll. Accordingly, the tissue simulation structure 102 and channel structure 108 may be designed to permit the channel 106, i.e., the simulated vein, to roll 1 millimeter, up to 2 millimeter or more. The simulated vein roll may generally occur in the transverse direction. The composite structure of the tissue simulation structure 102 may be formed to simulate a variety of different vein roll amounts. For example, the tissue simulation structure 102 can be formed to permit the channel 106 to “roll” 0.2 mm to simulate a person less prone to vein roll, or the tissue simulation structure 102 can be designed to permit the channel 106 to roll 1.0 mm to simulate a person more prone to vein roll. Additionally, channel 106 may also compress upon palpation of the tissue simulation surface by a user.

Medical simulation device 100 is designed to provide interactive training for administering intravenous injections, blood draws, and insertion of catheters. The channel 106 of medical simulation device 100 is designed to simulate a blood vessel and accordingly to receive a needle. A user may insert a needle into the channel 106, i.e., the simulated blood vessel, by inserting a needle through the tissue simulation surface of the tissue simulation structure 102.

Upon entry of the needle, the channel 106 may generate flash back in the needle. This provides a visual indicator to the user that the needle was properly inserted into the simulated vein. The channel 106 may also provide a portion of the fluid from channel 106 and compressible reservoir 104 to the needle, for example, if the user is attempting to simulate a blood draw. The channel 106 may also receive an external fluid via the inserted needle. For example, channel 106 may receive a saline flush, water, simulated drug, e.g., simulated clotting factor concentrate, or other suitable fluid administered through the inserted needle. The external fluid may then mix with the blood simulation fluid within the channel 106 and be contained by the compressible reservoir 104.

In order to protect from accidental needle pricking of a user, a protective layer may be attached to a bottom surface of the medical simulation device 100. The protective layer can limit penetration of the needle through the medical simulation device 100 to ensure that a user cannot insert the needle too far, at the incorrect angle, or otherwise improperly such that it extends beyond the medical simulation device 100 and injures the patient.

Upon withdrawal of the needle, as described previously, the tissue simulation surface may self-seal, ensuring that there are no visible indicators of previous injection sites in the medical simulation device 100 and sealing channel 106 may self-seal, ensuring that no fluid leaks out.

Medical simulation device 100 is intended to enable users to learn the techniques of, and gain experience with, locating blood vessels and administering intravenous injections, blood draws, and insertion of catheters in a safe and controlled manner. Instead of practicing on the actual human body, users are provided a realistic simulated experience using medical simulation device 100. Previous approaches utilize bulky simulators, mannequins, and/or simulated arms, which can be bulky, expensive, not easily configurable, and do not provide a realistic experience of administering an intravenous injection on a user's own body. The embodiments described herein may help overcome such drawbacks by providing a simplified at-home simulation device to learn how to perform and self-administer intravenous injections, blood draws, and insertion of catheters.

Medical simulation device 100 provides a relatively low-profile simulation device that can be readily placed on a human hand, arm, or other body part such that a user can gain experience and train in self-administering intravenous injections, blood draws, and insertion of catheters with realistic tissue appearance and function and tactile responsiveness and feedback. As described previously, the material properties of medical simulation device 100 may be varied to match different users, providing a more realistic experience tailored to each particular user. Additionally, medical simulation device 100 may be 3D printed, permitting it to be manufactured for relatively low cost.

Medical simulation device 100 allows users to learn, train, and gain experience with a variety of techniques. Users may gain experience with anatomy identification, in particular, with blood vessel palpations. The tissue simulation surface permits a user to gain experience with locating the vein, i.e., channel 106, with realistic tactile feedback. In using medical simulation device 100, a user may also gain experience with vein roll and other internal movement of the blood vessel within the tissue simulation structure 102. Medical simulation device 100 also permits users to gain experience with needle insertion, injection, blood flashback, saline flush, and needle removal.

Medical simulation device 100 can be used by a wide variety of users. Medical students, trainees, professionals, and others might use medical simulation device 100 to learn proper palpation techniques, intravenous injection techniques, and the like, or continue to practice and refine their skills in such techniques. Medical simulation device 100 can be employed in a wide variety of settings, including in educational or medical training sessions, or even in hospitals on patients. For example, if a patient is experiencing hesitation with receiving an intravenous injection, a nurse, doctor, or other medical professional might use the medical simulation device 100 on the patient, e.g., on their arm, to demonstrate how the operation is performed, to help make the patient feel more comfortable.

Medical simulation device 100 may be particularly useful for users to learn and practice in-home treatment. In-home treatment includes the intravenous administration of drugs or other fluids in non-medical settings. For example, medical simulation device 100 may be useful to hemophilia patients, especially children and adolescents. Oftentimes these patients must self-administer intravenous treatments on a regular basis. Accordingly, it is important they learn the proper techniques to perform intravenous injections, including through the use of a medical simulation device, before actually performing the treatments on themselves. In addition, parents, guardians, or other caregivers may use medical simulation device 100 to learn such techniques. Medical simulation device 100 provides a safe and controlled experience.

Medical simulation device 100 may be used in a variety of manners. Users may practice using the medical simulation device 100 as a standalone device, e.g., with it resting on a benchtop, table, or other flat, non-human or human-model surface. Users may practice in this manner, for example, when they are first learning blood vessel palpation techniques and/or intravenous injection techniques. Users may also practice with the medical simulation device 100 by placing it on another device that is meant to simulate human anatomy, e.g., a model arm or hand.

Medical simulation device 100 can also be used to practice directly on a patient, whether a user is practicing on themselves as the patient, or another person. The medical simulation device 100 may be placed on a person's body, e.g., on the user's arm or hand. In practicing on themselves, a patient-user may place medical simulation device 100 at the location where they will have to self-administer intravenous injections in the future. This provides the patient-user a realistic visual experience, i.e., how it looks to self-administer an intravenous injection, and also a realistic tactile experience, i.e., how it feels to palpate the tissue to locate the blood vessel and how to perform it using only one hand.

An example method of using the medical simulation device 100 in such a manner is further described. A patient-user practicing the techniques of blood vessel palpation and intravenous injection administration places the medical simulation device 100 on their body, e.g., their arm. The medical simulation device 100 may be placed at a location such that the tissue simulation structure 102 is the location where the patient-user will eventually self-administer intravenous injections.

The patient-user then places a tourniquet, such that the tourniquet is in contact, and generally covering, the compressible reservoir 104. In the case of a pneumatic tourniquet, the patient-user may then pressurize the tourniquet, applying an external pressure to the compressible reservoir 104. In the case of an elastic tourniquet, the user may tighten the tourniquet such that it applies an external pressure to the compressible reservoir 104.

As the compressible reservoir 104 is compressed, the fluid within the compressible reservoir 104, and accordingly the fluid in channel 106, becomes pressurized. Pressurizing of the fluid within the medical simulation device 100 may then simulate occlusion of the simulated blood vessel, causing channel 106 to swell or bulge due to the rise in pressure. The swelling of channel 106 generally simulates what will occur in the patient-user's actual body upon application of a tourniquet, thus providing a realistic experience.

Once the tourniquet is applied, and the fluid within compressible reservoir 104 and channel 106 is pressurized, the patient-user may then palpate the tissue simulation surface of the tissue simulation structure 102. As described previously, the tissue simulation surface provides realistic tactile responsiveness and feedback such that the patient-user will learn the techniques necessary before performing the operation on their actual body.

Once the patient-user has located the “vein,” i.e., channel 106, the patient-user may insert an intravenous needle through the tissue simulation surface and into channel 106. If the patient-user inserts the needle properly, channel 106 should provide flashback, i.e., confirmation of the needle placement through a visual indicator in the needle. The patient-user may then insert the catheter and/or administer a simulated injection, e.g., saline flush, to practice administering an injection. Upon completion of inserting the catheter and/or administering the simulated injection, the user may remove the needle from channel 106 through the tissue simulation surface. As described previously, the tissue simulation surface may then self-seal, ensuring that no fluid is released from the medical simulation device 100 and the medical simulation device can be used again and again.

By practicing with the medical simulation device 100, a user may learn the proper palpation and intravenous injection techniques, and also become more comfortable with the visual and tactile experiences for performing at-home intravenous injections. Medical simulation device 100 can provide a realistic, reusable, training system to help at-home users self-administer intravenous injections.

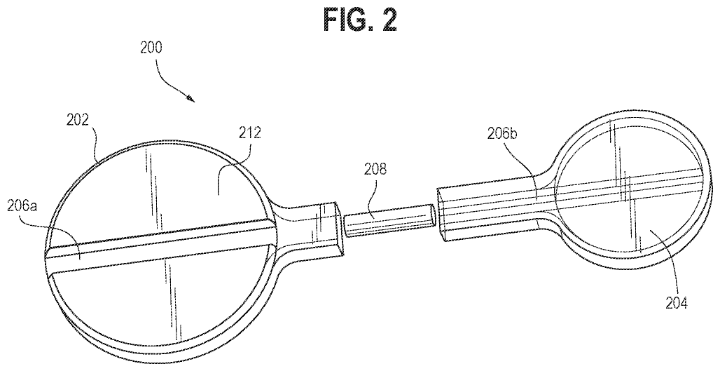

FIG. 2 is a perspective view of a second embodiment of a medical simulation device 200 in accordance with certain aspects of the present disclosure. The medical simulation device 200 may include several components, including tissue simulation structure 202 (which includes tissue simulation surface 212), compressible reservoir 204, and channel structure 208. As depicted in FIG. 2, the components may be separable. The tissue simulation structure 202, compressible reservoir 204, and channel structure 208 may be separately formed using additive manufacturing, e.g., 3D printing. After finishing the formation of each individual component, the tissue simulation structure 202, compressible reservoir 204, and channel structure 208 may be assembled to form the medical simulation device 200. The tissue simulation structure 202, compressible reservoir 204, and channel structure 208 may also be separately formed using conventional manufacturing and similarly assembled.

As depicted in FIG. 2, the channel forming the simulated blood vessel of medical simulation device 200 may include several portions, including a first channel portion 206a and a second channel portion 206b. The first channel portion 206a may generally be formed within the tissue simulation structure 202. The second channel portion 206b may generally be formed within the compressible reservoir 204. The first channel portion 206a may be in fluid communication with the second channel portion 206b via channel structure 208 that also includes a channel portion (not shown in FIG. 2). As described previously with reference to FIG. 1, the external shape of channel structure 208 may take a variety of different forms.

Channel structure 208 may be assembled with tissue simulation structure 202 and compressible reservoir 204 to form the medical simulation device 200. The channel structure 208 may be of various sizes to accommodate various assemblies of the medical simulation device 200. In some examples, as depicted in FIG. 2, the outer dimension of the channel structure 208 may be sized such that it can slide within an internal dimension of the channel portion 206a within operation simulation structure 202 and/or an internal dimension of the channel portion 206b within the compressible reservoir 204. The channel portions 206a and 206b may include a counterbore, countersink, or other suitable shoulder that ensures the internal dimension of channel structure 208 is similar to the internal dimension of the channel portions 206a and 206b.

In other examples, the inner dimension of channel structure 208 may be sized such that it fits over an outer dimension of tissue simulation structure 202 and/or compressible reservoir 204. The channel structure 208 may include a counterbore, countersink, or other shoulder such that the channel structure 208 has two inner dimensions. The larger inner dimension of channel structure 208 may be sized such that it fits over an outer dimension of tissue simulation structure 202 and/or compressible reservoir 204 while the smaller inner dimension of channel structure 208 may be sized to generally be a similar dimension of the first channel portion 206a and the second channel portion 206b.

The components forming medical simulation device 200 may be assembled such that they are permanently affixed. For example, in assembling the medical simulation device 200, channel structure 208 may be adhered using a suitable adhesive to the tissue simulation structure 202 and/or compressible reservoir 204. The components forming medical simulation device 200 may also be temporarily affixed. For example, the tissue simulation structure 202 and the compressible reservoir 204 may include barbed fittings such that the channel structure 208 fits over the barbs. In other examples, channel structure 208 includes the barbed fittings and the tissue simulation structure 202 and the compressible reservoir 204 include portions to fit onto channel structure 208.

Including temporary connections in the medical simulation device 200 permits it to be disassembled. In some examples, compressible reservoir 204 is a reloadable cartridge. The reloadable cartridge may be a single-use, prefilled cartridge. The cartridge may be pre-filled with a fluid, e.g., blood simulation fluid, such that a user does not have to fill the cartridge themselves. The reloadable cartridge may include a seal for the fluid that is removed by a user prior to assembly. Optionally, a seal in the reloadable cartridge may be self-removed/broken upon proper assembly into the medical simulation device 200. In some examples, the reloadable cartridge may be multi-use, permitting a user to empty and refill the cartridge, then reassemble with the medical simulation device 200.

Use of several assembled components, as depicted in FIG. 2 may help extend the life of the medical simulation device 200. For example, if upon a high number of training exercises using the medical simulation device 200, the tissue simulation surface 212 begins to degrade, e.g., needle holes become visible, or fluid-filled pockets lose fluid, the degraded component can be swapped out for a new component. This may help reduce waste and extend the usable life of the medical simulation device 200.

Additionally, by using assembled components, various tissue simulation structures 200 may be used to simulate different use scenarios. For example, if the medical simulation device is used within a healthcare setting where different users practice, various tissue simulation structures 202 may be swapped out such that the color tones of the tissue simulation surface 212 match the user's skin tone that is practicing. In this way, a single compressible reservoir 204 may be used with multiple tissue simulation structures. As another example, various tissue simulation surfaces of the tissue simulation structure 202 may be used to simulate various parts of the body, e.g., a first tissue simulation structure may simulate the back of the hand, while a second may simulate the inside of the elbow. Again, this permits various simulation scenarios while using a compressible reservoir 204 and/or channel structure 208.

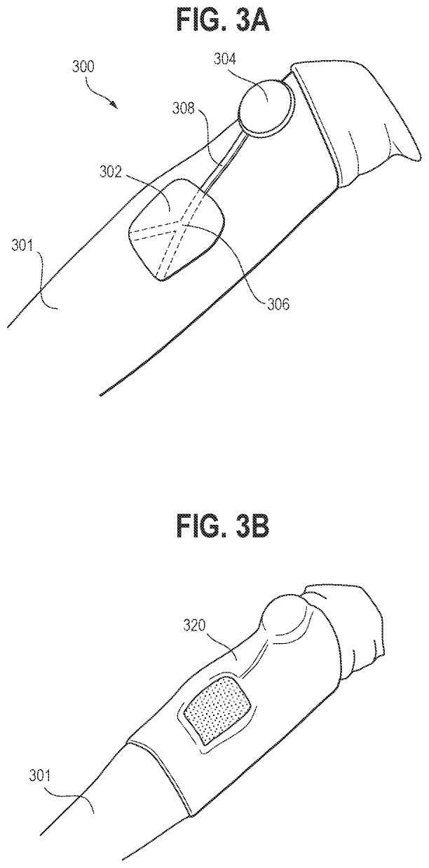

FIGS. 3A and 3B are depictions of a third embodiment of a medical simulation device 300 in accordance with certain aspects of the present disclosure. As previously described, the medical simulation device 300 may include a tissue simulation structure 302 which includes a tissue simulation surface, a channel structure 308, a compressible reservoir 304, and a channel 306 in fluid communication with the compressible reservoir 304.

In order to provide interactive training for intravenous injections, the medical simulation device 300 may be placed on a body part 301. Body part 301 may be an actual human body part, and more specifically, a body part of the patient-user training with the medical simulation device 300. Thus, as an example, and as is depicted in FIG. 3, the patient-user may place the medical simulation device 300 on the inside elbow of their arm. The patient-user may then place a tourniquet on their arm, generally in contact with compressible reservoir 304, pressurizing the fluid in compressible reservoir 304 and channel 306. The patient-user may then use their other hand to palpate the tissue simulation surface of tissue simulation structure 302 to determine the location of the channel 306 simulating the blood vessel. Upon locating the channel 306 via palpation, the patient-user may then practice inserting an intravenous needle through the tissue simulation surface into channel 306.

Body part 301 may also be a simulated body part, e.g., simulated arm, hand, leg, etc. Thus, as an example, a user may place the medical simulation device 300 on the simulated body part 301 to practice. The user may generally follow the same process described above, though instead of only being able to use one hand, the user may be able to practice using both hands. Users may train with the medical simulation device 300 on a simulated body part 301, for example, when they are first learning how to administer intravenous injections, such that they can learn with full use of their hands before training how to administer on themselves using only a single hand.

Although not depicted in FIG. 3A, the medical simulation device 300 may also include an adhesive layer. The adhesive layer may be attached to a bottom surface of the tissue simulation structure 302, i.e., beneath the channel 306. The adhesive layer may adhere the medical simulation device 300 to the skin tissue or simulated skin tissue of body part 301. The adhesive layer may generally hold the medical simulation device 300 in place on the body part 301, preventing lateral or vertical movement.

As depicted in FIG. 3B, the medical simulation device 300 may include a covering layer 320. The covering layer 320 may prevent movement of the medical simulation device 300 on the arm, hand, simulated body part, or other suitable location. The covering layer 320 may be a sleeve that fits over the medical simulation device 300 and the simulated body part 301. In some examples, the covering layer 320 may be a sleeve that the medical simulation device 300 fits within, e.g., the sleeve includes a pocket for the medical simulation device 300. The sleeve may include elastic or other suitable material such that it forms a generally tight fit around the medical simulation device 300 and body part 301 to hold in place and constrain movement of the medical simulation device 300 on the body part 301. The covering layer 320 may be a different color than the body part 301, or may generally match such that it blends with the color tone of the skin of the body part 301 and tissue simulation surface of the medical simulation device 300.

The covering layer 320 may also be an adhesive layer attached to the tissue simulation surface of the tissue simulation structure 302, i.e., above the channel 306. The adhesive layer may adhere the medical simulation device 300 to the skin tissue or simulated skin tissue of body part 301. The adhesive layer may generally hold the medical simulation device 300 in place on the body part 301, preventing lateral or vertical movement.

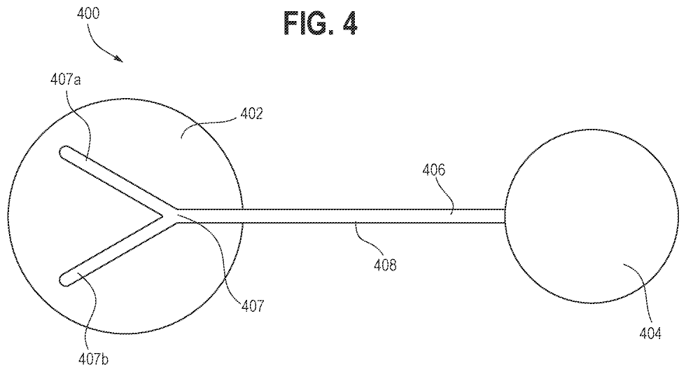

FIG. 4 is an illustration of a fourth embodiment of a medical simulation device 402 in accordance with certain aspects of the present disclosure. While the medical simulation device as depicted in FIG. 1 includes only a single simulated blood vessel, the medical simulation device may include multiple channels. The medical simulation device 400 as depicted in FIG. 4 shows one such embodiment. The medical simulation device 400 includes a tissue simulation structure 402, a compressible reservoir 404, and a channel structure 408 defining a channel 406.

The medical simulation device 400 may simulate multiple blood vessels. As depicted in FIG. 4, the channel 406 may include a branched portion 407. The branched portion 407 may form a first channel branch 407a and a second channel branch 407b. The first channel branch 407a may simulate a first blood vessel, and the second channel branch 407b may simulate a second channel branch. Although the branched portion 407 is depicted in FIG. 4 as forming two channel branches, the branched portion 407 may include two or more channel branches. The two or more channel branches accordingly may simulate two or more blood vessels.

The branched portion 407 may be entirely within the tissue simulation structure 402, i.e., beneath the tissue simulation surface, or the branched portion 407 may be outside of the tissue simulation structure 402. For example, the channel 406 may branch prior to entering the tissue simulation structure 402. In such examples, the first channel branch 407a and second channel branch 407b may pass beneath the tissue simulation surface of the tissue simulation structure 402 entirely separately.

In some examples, the medical simulation device 400 may include two or more channels 406. The two or more channels 406 may each independently be in fluid communication with the compressible reservoir 404. The two or more channels 406 may each simulate different blood vessels. Optionally, medical simulation device 400 may include two or more compressible reservoirs 404, each in fluid communication with the separate two or more channels 406.

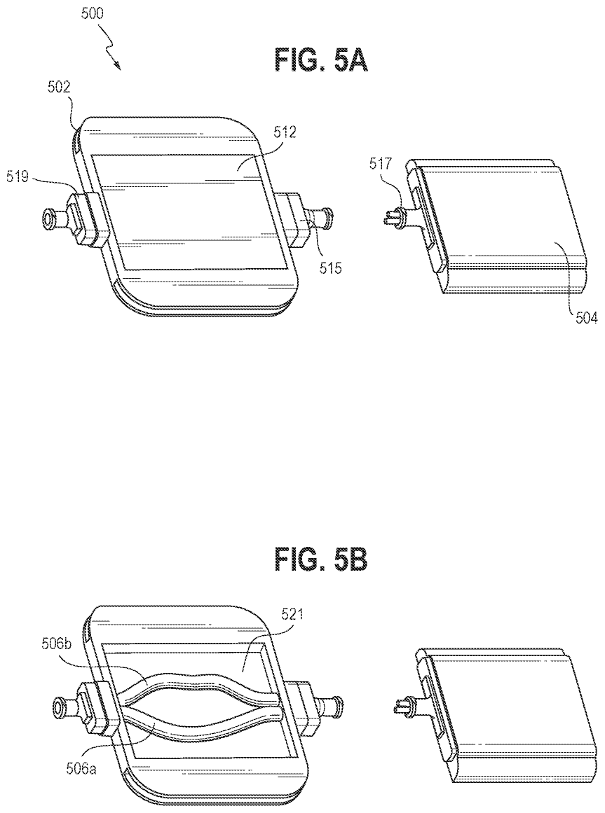

FIGS. 5A-5B are depictions of a third embodiment of a medical simulation device 500 in accordance with certain aspects of the present disclosure. As previously described, the medical simulation device 500 may include a tissue simulation structure 502 which includes a tissue simulation surface 512, and a compressible reservoir 504. The tissue simulation structure 502 may be connected to the compressible reservoir 504 using various types of connections and/or connectors. This connection may form a channel structure. For example, tissue simulation structure 502 may include a connector portion 515. This connector portion 515 may be connected to a connector portion 517 of the compressible reservoir 504. This connection may be direct, or may include one or more intermediate components, such as one or more channel segments, e.g., tubing segments. The connector portion 515 and connector portion 517 may be any suitable type of connector or fitting suitable for fluids. In some examples, the connector portion 515 and connector portion 517 may be leur type connectors. The tissue simulation structure 502 may also include a second connector portion 519. The second connector portion 519 may be the same type of connector or fitting as connector portion 515, or a different type. The second connector portion 519 may also be a type of valve. In some examples, second connector portion 519 is a stopcock.

In some examples, the medical simulation device 500 may be received by a user in an unfilled state, i.e., without any fluid in the compressible reservoir 504 or the tissue simulation structure 502. A user may fill the medical simulation device 500 with the fluid. A syringe (not depicted) may be connected to the second connector portion 519. The syringe may then be used to inject a fluid into the medical simulation device 500, filling the compressible reservoir 504. Once the medical simulation device 500 has received a sufficient amount of the fluid, the second connector portion 519 may be closed off such that the fluid is retained within the medical simulation device 500. In some examples, the second connector portion 519 may be a stopcock. In other examples, the syringe may be connected to directly to the compressible reservoir 504 via the connector portion 517 to fill the medical simulation device 500 with the fluid. After filling the compressible reservoir 504 with the fluid, the compressible reservoir 504 may be fluidly connected to the tissue simulation structure 502.

The tissue simulation structure 502 may include two or more channels, depicted in FIG. 5B as 506a and 506b, beneath the tissue simulation surface 512. The channels 506a and 506b may each simulate different blood vessels. The tissue simulation structure 502 may also include a protective layer 521. The protective layer 521 may help prevent accidental needle pricking of a user. The protective layer 521 may be attached to a bottom surface of the tissue simulation structure 502. The protective layer 521 may help ensure that a user cannot insert the needle too far, at the incorrect angle, or otherwise improperly such that it extends through the tissue simulation structure 502 an injures the user-patient. The protective layer 521 may be formed as a unitary part of the tissue simulation structure 502, or may be a separate component attached to the tissue simulation structure 502.

In some examples, some portion of the tissue simulation structure 502 may be a hollow or open space. For example, the space between the protective layer 521 and the tissue simulation surface 512 that surrounds the channels 506a and 506b may be hollow or open portion. In some examples, this hollow or open space may be filled using one or more various materials including, for example, an elastomeric material such as an organic or silicone-based molecule, latex, polymeric material, plastic, gel, and/or viscous fluid. This portion may also be filled using a two-part urethane, a semi-rigid material such as polyethylene, a fluid material, or other suitable material. The material may be selected to have a density and/or other properties that are similar to the corresponding anatomical tissue. The tissue simulation structure 502 may be formed of one or more such materials. In some examples, the tissue simulation structure 502 may include a fibrous layer, such as a nylon mesh to correspond to anatomical tissue.

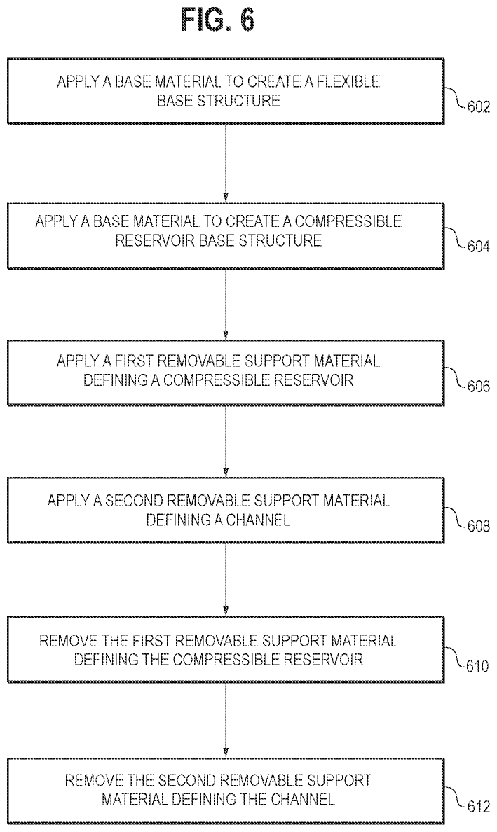

FIG. 6 is a flow diagram of an example method for the manufacture of a three-dimensional printed device in accordance with certain aspects of the present disclosure. To assist in the understanding, certain reference may be made to the reference numerals and components as shown and described in FIG. 1, though any of the embodiments shown or described in FIGS. 1-5 may be manufactured using the method of FIG. 6. As described with reference to FIGS. 1-5, the medical simulation device used to provide interactive training for various medical operations, including administering intravenous injections, may be formed using 3D printing.

Accordingly, at 602, a base material may be applied to create a flexible base structure. The flexible base structure may form, in part or in whole, the tissue simulation structure 102 as shown and described with reference to FIG. 1. The flexible base structure may also form, in part or in whole, the channel structure 108. The base material may include one or more component materials. For example, as described with reference to FIG. 1, the tissue simulation structure may include fluid-filled pockets. Accordingly, at 602, a first material may be applied forming the composite structure, and a second material may be applied filling the fluid-filled pockets.

At 604, a compressible material may be applied to create a compressible reservoir base structure. As described with reference to FIG. 1, the application of the compressible material may create, in part or in whole, compressible reservoir 104. The compressible material may be the same material as the base material, or it may be different. For example, the base material may be chosen to generally match the tactile responsiveness of skin tissue, while the compressible material may be chosen to optimize fluid pressurization.

As shown and described with reference to FIG. 1, because the compressible reservoir 104 includes a cavity to contain a fluid, when 3D printing the component, support material may be necessary to define the internal structure of the compressible reservoir. Accordingly, at 606, a removable support material may be applied to the compressible reservoir base structure. The removable support material may define a cavity, i.e., the internal structure, of the compressible reservoir. In some examples, the removable support material defining the cavity of the compressible reservoir may be applied all at once. In other examples, the removable support material may be applied in stages. For example, as a given layer of the 3D printed device is applied, a portion of the removable support material may be applied, then a portion of the compressible material may be applied. As the 3D printer moves through each layer, additional removable support material and compressible material may be applied, in alternating or sequential order, to create the compressible reservoir. Upon fully applying the removable support material and the compressible material, a fully formed compressible reservoir may be formed that can contain a fluid (after a subsequent removal of the removable support material).

As shown and described with reference to FIG. 1, because the channel 106 is in fluid communication with the compressible reservoir 104, at 608, removable support material may be applied to the flexible base structure to define the channel 106, i.e., the internal structure of the channel structure 108. The channel structure 108 may be formed of the base material, or may be formed of a different material. As described above, in some examples, the support material defining the channel may be applied all at once. In other examples, the support material may be applied in stages. For example, as the 3D printer applies each layer, a portion of the removable support material may be applied in addition to a portion of the base material. The 3D printer may move to the next layer and repeat this process. Upon fully applying the removable support material and the base material, a fully formed channel structure, including the channel may be formed that can contain a fluid and is in fluid communication with the compressible reservoir and extends through a portion of the flexible base structure.

The application of base material, compressible material, and support material may be repeated until a fully formed medical simulation device is formed, as shown and described with reference to FIGS. 1-4. As should be appreciated, no particular order of the steps is required. For example, the compressible material may be applied before the base material (i.e., step 604 can be performed before step 602), or the removable support material defining the channel may be applied before the removable support material defining the cavity of the compressible reservoir (i.e., step 608 can be performed before step 606).

In addition, steps may be added and are within the scope of the present disclosure. For example, in 3D printing the medical simulation device, support material may be applied first, before applying the base material at 602. Optionally, a third material may be applied, e.g., a material forming the channel structure, that is different from the base material applied at 602. This may be performed, for example, prior to step 608.

Steps may also be combined and are within the scope of the present disclosure. As an example, step 602 and step 604 may be combined and performed concurrently, e.g., when the base material and compressible material are formed using the same material. As another example, step 606 and step 608 may be combined and performed concurrently, e.g., when the same removable material is used to define the compressible reservoir and to define the channel.

At the optional 610, the reservoir support material may be removed. The support material defining the compressible reservoir may be removed after the device is fully 3D printed, or it may be removed part-way through the 3D printing of the device. As shown and described with reference to FIG. 1, the reservoir support material 114 may be removed though an access port of the compressible reservoir 104.

At the optional 612, the channel support material may be removed. The support material defining the channel may be removed after the device is fully 3D printed, or it may be removed part-way through the 3D printing of the device. As shown and described with reference to FIG. 1, the channel support material 116 may include a tabbed portion and an elongate portion. The elongate portion may define the channel 106. By pulling on the tabbed portion of the channel support material, the elongate portion may be removed, defining the channel 106. The channel support material 116 may be removed through an access port of the compressible reservoir 104.

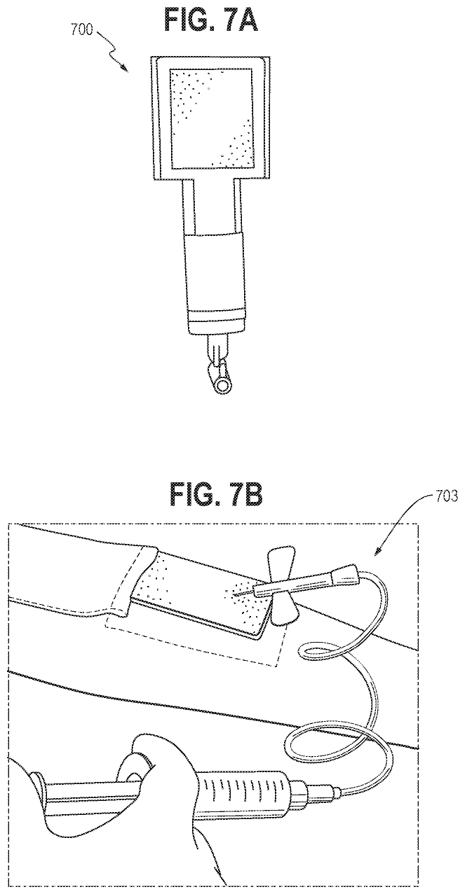

FIGS. 7A-7C depict a non-limiting prototype of a device having features discussed herein. The medical simulation device, depicted in FIG. 7A may be placed into a sleeve that is placed on a simulated arm, or an actual user's arm. The tissue simulation structure may be exposed, simulating skin on the user, while the compressible reservoir may be covered by a portion of the sleeve, as shown in FIGS. 7A and 7B. The user may then practice proper palpation techniques, intravenous injection techniques, and the like using the medical simulation device, while located on the user's arm.

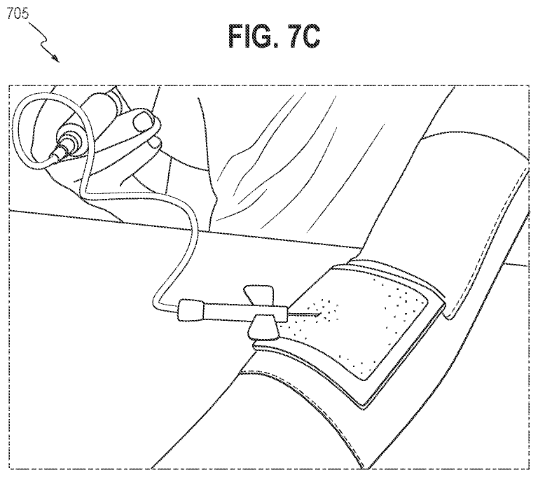

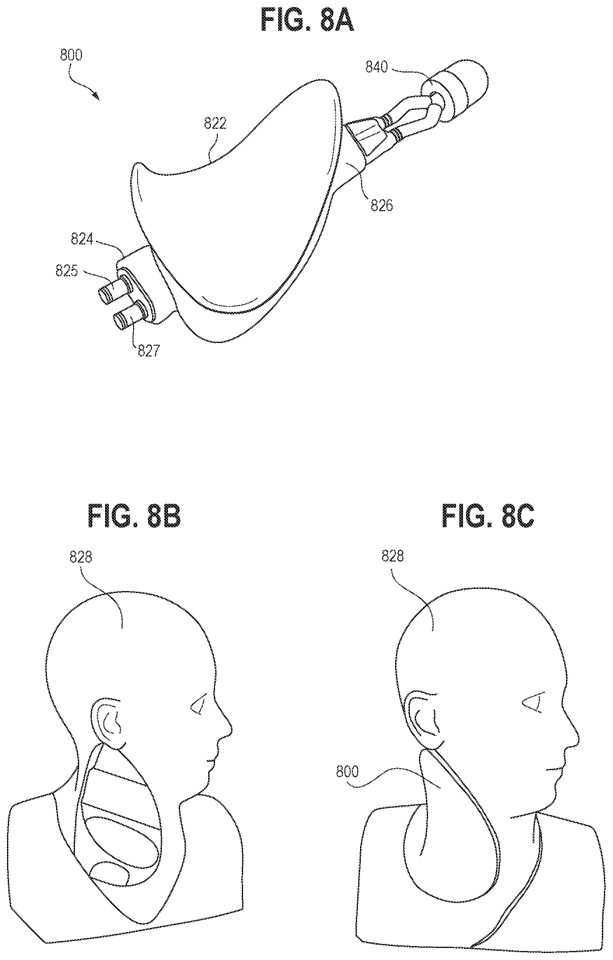

FIG. 8A is a perspective view of a sixth embodiment of a medical simulation device 800 in accordance with certain aspects of the present disclosure.

Medical simulation device 800 may be produced using additive manufacturing, and in particular, 3D printing. All, or portions of, medical simulation device 800 may also be produced using conventional manufacturing processes, including forming, molding, or other suitable process. Medical simulation device 800 includes a tissue simulation structure 822. Medical simulation device 800 may also include a first connector portion 824 and a second connector portion 826. In some examples, medical simulation device 820 includes a flow restrictor 840. The flow restrictor 840 may be connected to the second connector portion 826.

Medical simulation device 800 may be used to provide training for various medical operations, including central line catheter insertions and peripherally inserted central line catheters, by simulating a human blood vessel within simulated human bodily tissues. The simulated blood vessel may be designed to simulate the internal or external jugular vein, subclavian vein, femoral vein, or other vein within the body. The simulated blood vessel may also be designed to simulate a human artery. Medical simulation device 800 allows a user to learn the proper techniques of blood vessel identification and venous catheter insertion.

The tissue simulation structure 822 may have the varied structures and/or properties as those of tissue simulation structure 102 as described with reference to FIG. 1. Tissue simulation structure 822 may be formed via 3D printing. In some examples, the tissue simulation structure 822 may be 3D printed to have a texture that is similar to the human body. The tissue simulation structure 822 may be formed such that it feels and looks like human tissue, including skin tissue, muscle tissue, fat tissue, or subcutaneous tissue. In some examples, the tissue simulation structure 822 includes a formed channel that simulates a blood vessel. The formed channel may be formed via additive manufacturing, e.g., 3D printing. In other examples, the tissue simulation structure 822 includes tubing or other similar, pre-made channel to simulate a blood vessel that is integrated within the tissue simulation structure 822. The channel is designed to simulate a blood vessel and accordingly to receive a needle and/or a catheter. The channel may be in fluid communication with a reservoir comprising a fluid.

The structure forming tissue simulation structure 822 is designed to simulate the feel and tactile feel of various types and/or areas of human tissue. For example, the properties of tissue simulation structure 822 may be varied to simulate the tissue on the neck, upper chest, thigh, or other location corresponding to the insertion point on a human body. The properties of tissue simulation structure 822 may also be varied such that the simulated tissue is similar to the tissues of a specific type of individual.

In some examples, some portion of the tissue simulation structure 822 may be a hollow or open space. For example, the medical simulation device 800 depicted in FIG. 8A may include a tissue simulation structure 822 that includes a tissue simulation surface with one or more channels beneath the tissue simulation surface. The volume surrounding the one or more channels beneath the tissue simulation surface may otherwise be open. In some examples, this hollow or open space may be filled using one or more various materials including, for example, an elastomeric material such as an organic or silicone-based molecule, latex, polymeric material, plastic, gel, and/or viscous fluid. This portion may also be filled using a two-part urethane, a semi-rigid material such as polyethylene, a fluid material, or other suitable material. The material may be selected to have a density and/or other properties that are similar to the corresponding anatomical tissue. The tissue simulation structure 822 may be formed of one or more such materials. In some examples, the tissue simulation structure 822 may include a fibrous layer, such as a nylon mesh to correspond to anatomical tissue.

As depicted in FIG. 8A, the medical device 800 may include a first connector portion 824. The first connector portion 824 may be any suitable type of connector or fitting suitable for fluids. For example, the first connector portion 824 may be a barb fitting, threaded fitting, or compression fitting. In some examples, the first connector portion 824 may include a coupling fitting. The first connector portion 824 may be a female portion of a connector or male portion of a connector. If the first connector portion 824 includes more than one port, the first connector portion 824 may also be comprised of a variety of male and female connector portions. In some examples, the first connector portion 824 may be a unitary structure and include a both an inlet port and outlet port. In other examples, the first connector portion 824 may include a first structure functioning as an inlet port 825, and a separate, second structure functioning as an outlet port 827. In some examples, the first connector portion 824 may include only a single port, functioning as either an inlet or an outlet. The second connector portion 826 may be the same as, or different from, the first connector portion 824.

The inlet port 825 of the first connector portion 824 may be in fluid communication via a channel with an outlet portion of the second connector portion 826. Similarly, in some examples, the inlet port of the second connector portion is in fluid communication via a channel with the outlet port 827 of the first connector portion 824. The outlet port and inlet port of the second connector portion 826 may be in fluid communication with each other via the flow restrictor 840.

FIGS. 8B-8C depict an example embodiment of the medical simulation device 800 being used to simulate the neck tissue of a human. As depicted in FIGS. 8B-8C, the medical simulation device 800 may be designed to fit within a defined portion of a human simulation structure 828. In other examples, the medical simulation device 800 is designed to fit on a surface of a human simulation structure. In some examples, the medical device 800 is formed such that it forms the human simulation structure 828 itself. Thus, the medical simulation device 800 may be formed such that it forms a structure that simulates a human head and neck area. As described previously, the medical simulation device 800 may simulate a human neck portion, chest portion, femoral portion, or other site used for the insertion of central line catheters, peripherally inserted central catheters, or other catheter type.

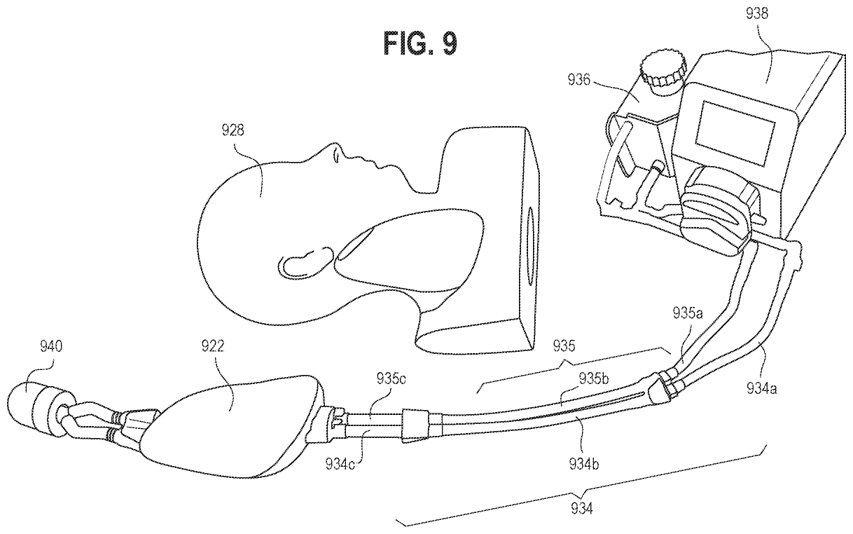

FIG. 9 is a depiction of an example setup of the medical simulation device 800 as described with reference to FIGS. 8A-8C. Similar to as described with reference to FIGS. 8A-8C, the medical simulation device 800 (comprised of tissue simulation structure 922 and flow restrictor 940) may be designed to fit within a human simulation structure 928. The tissue simulation structure 922 may be connected to, and in fluid communication with, a pump 938 via an inlet channel 934. The tissue simulation structure 922 may also be connected to, and in fluid communication with, a reservoir 936 via an outlet channel 935.

The inlet channel 934 may be connected to the pump 938 with one or more fittings or couplings. The outlet channel 935 may be connected to the reservoir 936 with one or more fittings or couplings. In some examples, the fittings or couplings include a valve, for example, a one-way check valve. The inlet channel 934 may be connected to the inlet portion of the first connector portion of the tissue simulation structure 922 to provide fluid pumped by the pump 938. The outlet channel 935 may be connected to the outlet portion of the first connector portion of the tissue simulation structure 922 to return fluid to the reservoir 936 after flowing through the tissue simulation structure 922 and the flow restrictor 940. In some examples, a single channel connects the tissue simulation structure 922 to an external component. For example, the tissue simulation structure 922 may be connected to the pump 938 via the inlet channel 934.

The channels 934 and 935 may include one or more segments, for example, as depicted in FIG. 9, channel segments 934a, 934b, and 934c, and 935a, 935b, and 935c. In some examples, each of the channels 934 and 935 includes more than three segments. In other examples, the channels 934 and 935 include fewer than three segments, e.g., one or two. The number of segments of the inlet channel 934 and the outlet channel 935 may be the same, or different. The channel segments, e.g., 934a, 934b, and 934c may be connected via one or more fittings or couplings. The various fittings and/or couplings of the channel segments, e.g., 934a, 934b, and 934c may include a valve, for example, a one-way check valve.

As depicted in FIG. 9, fluid pumped by the pump 938 may travel through the inlet channel 934, which passes within the tissue simulation structure 922. The pump 938 may produce pressure pulses to simulate a human heart's beating action. The pressure pulses of the fluid flowing through the inlet channel 934 may simulate a human artery within the tissue simulation structure 922. After the fluid flows through the inlet channel 934 and the tissue simulation structure 922, the fluid flows through the flow restrictor 940. As will be explained in further detail below, the flow restrictor 940 may reduce the pressure and pressure pulsations of the fluid. After flowing through the flow restrictor 940, the fluid may pass through a second channel in the tissue simulation structure 922. This may simulate a human vein within the tissue simulation structure 922. The fluid may then flow to the reservoir 936. In some examples, the reservoir 936 may be separate from pump 938 and form an open-loop system. The pump 938 may include a reservoir separate from the reservoir 936. In other examples, the reservoir 936 may be fluidly connected to the pump 938 to form a closed-loop and self-contained system such that the fluid can be repeatedly cycled through the system, including the tissue simulation structure 922.

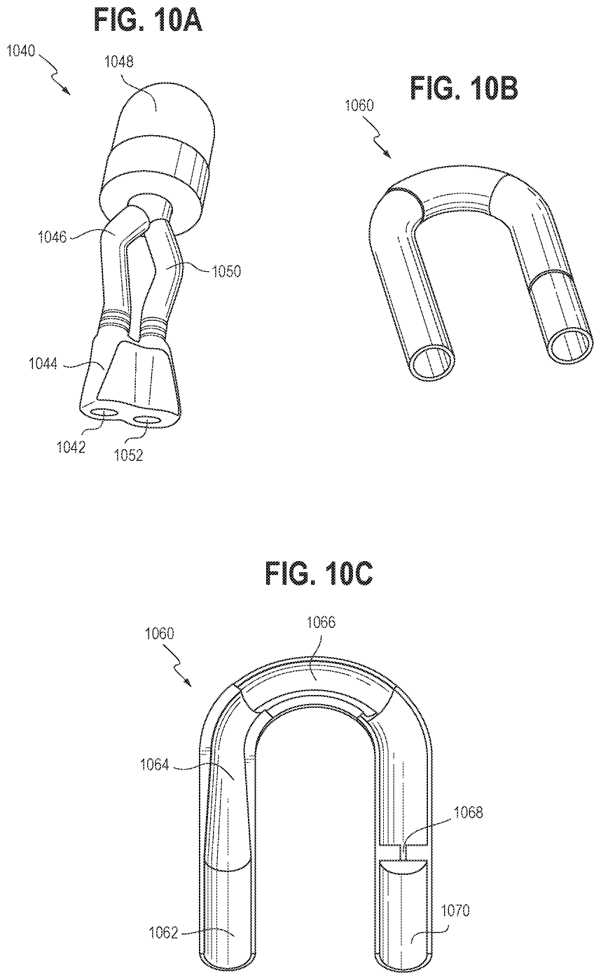

The flow restrictor of the medical simulation device 900 is shown in greater detail in FIG. 10A. Some reference numerals of FIG. 8A and FIG. 9 may be used while describing the flow restrictor 1040 for clarity. The flow restrictor 1040 may also be referred to as a pulse dampener. The flow restrictor 1040 may include a connector portion 1044. The connector portion 1044 of the flow restrictor 1040 may be connected to the second connector portion 826 of the medical simulation device 800. In some examples, the flow restrictor 1040 and the tissue simulation structure 822 may be formed as an integral, unitary structure, i.e., without the use of the second connector portion 826 and connector portion 1044.

The flow restrictor 1040 is in fluid communication with the tissue simulation structure 822. The connector portion 1044 of the flow restrictor 1040 may include an inlet 1042 and an outlet 1052. The inlet 1042 may receive fluid from the tissue simulation structure 822. From the inlet 1042, the fluid may flow through the inlet tube 1046 and through the flow restrictor portion 1048. After passing through the flow restrictor portion 1048, the fluid may flow through the outlet tube 1052 and flow through the tissue simulation structure 822 via the outlet 1052.

The flow restrictor 1040 may be configured to reduce a pressure and/or a pressure pulsation in the fluid, e.g., pressure pulsations produced by the pump 938. Similar to how a heart beats periodically that can create pressure pulsations in human blood, fluid pressure at the inlet 1042 of the flow restrictor 1040 may pulse because of repeated spikes in pressure produced by pump 938. These pulsations in the fluid channel may simulate a human blood vessel, e.g., an artery, in the tissue simulation structure 922. When the fluid flows through the flow restrictor 1040, the pressure and/or the pressure pulsations may be reduced due to the internal structures of the flow restrictor portion 1048, and explained in further detail below. When the fluid exits the flow restrictor 1040, the reduced pressure and/or pressure pulsations in the channel may simulate a human blood vessel, e.g., a vein, in the tissue simulation structure 922. The pressure and/or the pressure pulsations may be reduced because of the pulse dampener 1060 within the flow restrictor portion 1048.

FIGS. 10B-10C depict in more detail the internal structure of the flow restrictor portion 1048 of the flow restrictor 1040. The flow restrictor 1040 may be produced using additive manufacturing, and in particular, 3D printing. All, or portions of, the flow restrictor 1040 may also be produced using conventional manufacturing processes, including forming, molding, or other suitable process. The flow restrictor portion 1048 includes a pulse dampener 1060. The pulse dampener 1060 may include an inlet 1062 and an outlet 1070. The pulse dampener 1060 may include one or more features to regulate the pressure, pressure pulsations, and/or flow rate of a fluid. In some examples, the inlet 1062 of the pulse dampener 1060 includes a valve, e.g., a one-way check valve.

The pulse dampener 1060 may also include a flow reducer portion 1064. As depicted in FIG. 10C, the flow reducer portion 1064 may reduce a cross-sectional area of the fluid passage as compared to the inlet 1062 by narrowing the outer walls of the passage. In other examples, the flow reduce portion 1064 may include an obstruction to reduce a cross-sectional area of the passage. By reducing the cross-sectional area of the fluid passage, the flow volume of the fluid may be reduced.

The pulse dampener 1060 may include a compliant reservoir 1066. The compliant reservoir 1066 may be formed using a flexible material to permit it to deform and flex. By deforming and flexing, the compliant reservoir 1066 may reduce pressure pulsations in the fluid. The pressure pulsations in the fluid may be a result of the pumping action of the pump 938. The cross-sectional area of the compliant reservoir 1066 may be the same as the outlet of the flow reducer portion 1064, or may be different. For example, the compliant reservoir 1066 may have a larger cross-sectional area that the flow reducer portion 1064, allowing the compliant reservoir 1066 to further reduce pressure pulsations in the flow of the fluid.

The pulse dampener 1060 may include an orifice 1068. The orifice 1068 may significantly reduce the cross-sectional area of the fluid passage. In some examples, the orifice may reduce the inner diameter of the fluid passage by approximately 50-90%. In one example, the orifice 1068 may reduce the inner diameter of the fluid passage by over 80%, e.g., an inner diameter of the inlet 1062 is 8 mm and the diameter of the orifice is 1 mm. The orifice 1068 may extend along a length of the passage, or may be short in length, e.g., a plate. The cross-sectional shape of the orifice may also vary, and does not necessarily need to be a circle or cylindrical. For example, it may be ovular or conical in shape.

The orifice 1068 may reduce the pressure and/or pressure pulsations in the flow of the fluid. When the fluid passes through the orifice 1068, the pressure upstream of the orifice, e.g., in the compliant reservoir 1066, may increase slightly due to the flow restriction of the orifice 1068. After flowing through the orifice 1068, the pressure of the fluid in the outlet 1070 may be reduced because of pressure losses resulting from the obstruction. After flowing through the orifice 1068, pressure pulsations present in the fluid in the inlet 1062 may also be reduced in the outlet 1070. This reduction in the pressure and/or pressure pulsations may enable the tissue simulation structure 922 to simulate a human artery in the channel upstream of the pulse dampener 1060, and simulate a human vein in the channel downstream of the pulse dampener 1060.

Simulating blood vessels, including a human artery and human vein, in a tissue simulation structure can provide interactive training for medical professionals or other users for inserting and placing catheters, including central line catheters, peripherally inserted central line catheters, or peripheral intravenous catheters. The medical simulation device 800 may enable users to learn the techniques of, and gain experience with, locating blood vessels and inserting and placing catheters in a safe and controlled matter. Medical simulation device 800 can be used by a wide variety of users. Medical students, trainees, professionals, and others might use medical simulation device 800 to learn proper blood vessel identification techniques, catheter insertion and placements techniques, and the like, or continue to practice and refine their skills in such techniques. Medical simulation device 800 can be employed in a wide variety of settings, including in educational or medical training sessions. Instead of practicing on an actual human body, users are provided with a realistic simulated experience using medical simulation device 800.

While various embodiments have been described, it will be apparent to those of ordinary skill in the art that many more embodiments and implementations are possible. Accordingly, the embodiments described herein are examples, and not the only possible embodiments and implementations within the scope of this description.

Having described various aspects of the subject matter above, additional disclosure is provided below that may be consistent with the claims originally filed with this disclosure. In describing this additional subject matter, reference may be made to the previously described figures.

1. A medical simulation device comprising:
a tissue simulation surface for simulating an anatomical tissue surface;
a compressible reservoir configured to contain a fluid such that the fluid is pressurized when the compressible reservoir receives an external pressure; and,
a channel in fluid communication with the compressible reservoir, the channel passing beneath the tissue simulation surface such that the channel is accessible to receive a needle inserted through the tissue simulation surface.

2. The medical simulation device of claim 1, wherein the channel simulates at least one blood vessel.

3. The medical simulation device of claim 1, wherein the channel is configured to receive the needle when the needle is inserted through the tissue simulation surface.

4. The medical simulation device of claim 1 wherein the channel is located between the tissue simulation surface and a protective layer, wherein the protective layer is configured to limit penetration of the needle through a bottom surface of the medical simulation device.

5. The medical simulation device of claim 1, wherein the channel is configured to generate flash back when the needle is received.

6. The medical simulation device of claim 1, wherein the channel is configured to reseal upon withdrawal of the needle.

7. The medical simulation device of claim 1, wherein the tissue simulation surface is configured to reseal upon withdrawal of the needle.

8. The medical simulation device of claim 1, wherein the channel is configured to move in a transverse direction up to 2 millimeters to simulate vein roll.

9. The medical simulation device of claim 1, wherein the channel is configured to receive the fluid from the compressible reservoir when the compressible reservoir receives the external pressure, and wherein the compressible reservoir is configured to receive the fluid from the channel when the external pressure is released.

10. The medical simulation device of claim 1, wherein the fluid is a blood simulation fluid.

11. The medical simulation device of claim 1, the compressible reservoir further configured to collect a saline flush received through the needle.

12. The medical simulation device of claim 1, the compressible reservoir further configured to be removable from fluid communication with the channel to enable emptying and refilling of the compressible reservoir.

13. The medical simulation device of claim 1, wherein the channel comprises a branched portion forming a first channel branch and a second channel branch to simulate a first blood vessel and a second blood vessel.

14. The medical simulation device of claim 1, the medical simulation device further comprising:
a second channel in fluid communication with the compressible reservoir, the second channel extending away from the compressible reservoir and passing beneath the tissue simulation surface to simulate a second blood vessel.

15. The medical simulation device of claim 1, wherein the channel is configured to swell when the fluid is pressurized.

16. The medical simulation device of claim 1, the medical simulation device further comprising:
a compression sleeve covering the compressible reservoir and configured to constrain movement of the medical simulation device.

17. The medical simulation device of claim 1, wherein the anatomical tissue surface is at least one of a skin tissue, a muscle tissue, a fat tissue, and a subcutaneous tissue.

18. A device at least partially formed by additive manufacturing, the device comprising:
a flexible base structure;
a compressible reservoir configured to contain a fluid and to pressurize the fluid upon application of an external pressure; and,
a channel in fluid communication with the compressible reservoir, the channel extending away from the compressible reservoir and passing through at least a portion of the flexible base structure.

19. A method for the manufacture of a three-dimensional printed device, the method comprising:
applying a base material to create a flexible base structure;
applying a compressible material to create a compressible reservoir base structure;
applying a first removable support material to the compressible reservoir base structure, the first removable support material defining a cavity of a compressible reservoir, the compressible reservoir configured to contain a fluid and to pressurize the fluid upon application of an external pressure; and
applying a second removable support material to the flexible base structure, the second removable support material defining a channel, the channel extending from the compressible reservoir and extending through the flexible base structure.

20. The method of claim 19, the method further comprising:
removing the first removable support material defining the compressible reservoir; and,
removing the second removable support material defining the channel.
